(19) United States
(12) Patent Application Publication
(10) Pub. No.: US 2019/0380862 A1
(43) Pub. Date: Dec. 19, 2019

(54) ARM TENSIONING DEVICE TO PREVENT A SUPINE SLEEP POSITION

(71) Applicant: Gary FERDMAN, La Jolla, CA (US)

(72) Inventor: Gary FERDMAN, La Jolla, CA (US)

(21) Appl. No.: 16/436,178

(22) Filed: Jun. 10, 2019

Related U.S. Application Data
(60) Provisional application No. 62/684,941, filed on Jun. 14, 2018.

(51) Int. Cl. A61F 5/56; A61F 5/37

(52) U.S. Cl. CPC A61F 5/56; A61F 5/3723; A61F 5/3776

(57) ABSTRACT

A system for holding a sleeper in a non-supine (e.g.: side-lying) sleeping position by: (a) placing a wrist or arm of a sleeper into a cuff connected to one end of a band or tube of elastic material; (b) connecting the second end of the band of elastic material to a stationary object, thereby restraining the range of motion of the user's upwardly facing arm when sleeping; and then (c) having the sleeper sleep in the non-supine side-lying sleeping position with the cuff connected to the upper facing arm, such that tension on the cuff from the elastic material pulls on the user's arm and thereby prevents the sleeper from rolling onto their back.

### RELATED APPLICATION

The present application claims priority to U.S. Provisional Patent Ser. No. 62/684,941, of same title, filed Jun. 14, 2018, the entire disclosure of which is incorporated herein by reference in its entirety for all purposes.

### BACKGROUND OF THE INVENTION

According to the American Sleep Apnea Association, 90 million Americans suffer from snoring activity during sleep, and about half of them may have a serious sleep disorder called Obstructive Sleep Apnea (OSA).

Snoring can be reduced by avoiding the supine sleep position, and The American Journal of Respiratory and Critical Care Medicine indicates that the incidence and dangers of OSA can also be reduced by avoiding the “supine” (i.e.: lying on the back) sleep position.

Common treatments for OSA include facial masks, breathing devices, dental appliances, nerve stimulators, inconvenient and uncomfortable and often ineffective pillows, blocks of foam, vests with tennis balls or air bags and other devices and techniques that focus on the patient's head and/or back. All of these approaches are cumbersome, uncomfortable and many are inconvenient for travel.

In addition, the American Pregnancy Association recommends that the best sleep position during pregnancy is “SOS” (sleep on side), preferably the left side, which will increase the amount of blood and nutrients that reach the placenta. A 2017 study, published in the British Journal of Obstetrics and Gynaecology (BJOG) confirmed findings from smaller studies in New Zealand and Australia that the side sleep position during the third trimester substantially reduces the incidence of stillbirths.

Therefore, what is instead desired is a comfortable, inexpensive and easy to use system to keep a sleeper from rolling over onto their back and thus treat snoring and OSA. Ideally, such a system would also treat REM Sleep Behavior Disorder as well using this same approach. In addition, such a system would also be beneficial for pregnant women as it is safest for pregnant women to sleep on their (preferably left) side. Ideally, such a system would be easy to travel with and take up little space.

### SUMMARY OF THE INVENTION

The present supine sleep prevention wellness device is designed to enable the user to sleep comfortably on his or her side without rolling over onto their back during the night. The present system uses a band of elastic material to maintain tension on the sleeper's wrist (of the upwardly facing arm when the sleeper is sleeping on their side). Should the sleeper start to roll over onto their back, the added tension on the upwardly facing arm will make this movement uncomfortable. As such, the sleeper will instead adopt the more comfortable position of continuing to sleep on their side. The present system is based on patient ergonomics and physiology—specifically, it is difficult if not impossible to turn one's body 90 degrees (from side to back) if the sleeper's higher arm is constrained. (A person could conceivably rotate to a supine position by rotating 270 degrees in the other direction, but the present system would make this very difficult as well). As a result, the present system's elasticity, it's attachment to the wrist and anchoring and adjustment features make it a novel and useful apparatus. By gently restricting the range of motion of the sleeper's arm, the present system is comfortable, and not too confining. As such, it does not wake the sleeper to reposition him or her. This provides the benefit of a sound night's sleep.

By constraining arm and body motions, the present system may also help reduce the dangers of REM Sleep Behavior Disorder, a medically diagnosed condition in which the muscles are not fully paralyzed during sleep, so patients physically act out some of their dreams, often endangering themselves and/or their bedmates.

The present supine sleep prevention wellness device is designed to assure that the user sleeps on his or her side throughout the complete sleep cycle. In preferred embodiments, the system comprises a padded wrist (or arm) cuff that can have a diameter that is adjustable using hook and loop fastener materials (e.g.: Velcro®). Specifically, the user simply inserts his or her wrist into the cuff and adjusts the sizing of the cuff with a Velcro® strap. An elastic tube or band is connected to this cuff to provide tension on the upwardly facing arm (and thereby to prevent the user from rolling onto his or her back).

In one preferred aspect, the length of elastic material (which can be a tube or band or strap, etc.) can be looped back on itself and clamped to form an end loop. This elastic end loop can then be looped around a stationary object (e.g.: a night table or bed leg) and clamped back on itself to apply tension to the upwardly facing arm. In another preferred aspect, an end strap is attached to the elastic material to form an end loop, wherein the sizing of the end loop can be adjusted by passing an end of the strap back through a buckle. As such, the length of elastic material can be attached to a flat belt or strap and threaded through a buckle to adjust and secure its length.

In preferred embodiments, the elastic tubing comes in a kit with light, medium and heavy tension versions based on the arm strength of the user. The user then chooses the tension band/tube that is most appropriate for his or her own personal use. Preferably, the elastic tubing is composed of latex or synthetic rubber for users with latex allergies. In preferred methods of treatment, the present system pulls on the user's wrist at an angle of 45 to 135 degrees from the side of the bed. Preferably, for maximum effectiveness, the elastic tubing may pull in a direction perpendicular to the side of the bed.

The present wellness device system is comfortable and not confining unless the user attempts to move from the side position. Resistance increases the further the tubing is stretched, so, unlike current straps and belts, users have some mobility to enable falling asleep without feeling constrained. However, sufficient resistance is provided to enable the user to reach the supine position without sufficient tension on the arm to awaken them or make the motion impossible. Yet another advantage of the present system is that it is very easy to get out of in an emergency. In contrast, many existing systems hold sleepers so securely in one position that they become a safety hazard. Also, users often wake during the night to go to the bathroom. It is therefore advantageous to have a system like the present invention which can easily be removed and re-attached to the user (even in the dark).

### BRIEF DESCRIPTION OF THE DRAWINGS

FIG. 1 is an illustration of an embodiment of the present wellness device system having a clamp securing a loop at an end of the elastic material.

FIG. 2 is an illustration of an embodiment of the present system having a buckle securing a loop of a strap attached to an end of the elastic material.

FIG. 3 is an illustration of a user of the present system sleeping in a non-supine position with an end of the device anchored on a stationary object (e.g.: the leg of a night table).

FIG. 4 is an illustration of a user of the present system sleeping in a non-supine position with an end of the device anchored on a stationary object (e.g.: a side middle leg of a bed).

FIG. 5 is an illustration of a mechanism using a band and eyelet for anchoring the present system either to a mattress or box spring of a bed.

FIG. 6 is an illustration of a mechanism using a hook and loop fastener approach for anchoring the present system between the mattress and box spring of a bed.

FIG. 7 is an illustration of a mechanism using a clamp and spindle for anchoring the present system to a shelf adjacent to the bed.

### DETAILED DESCRIPTION OF THE DRAWINGS

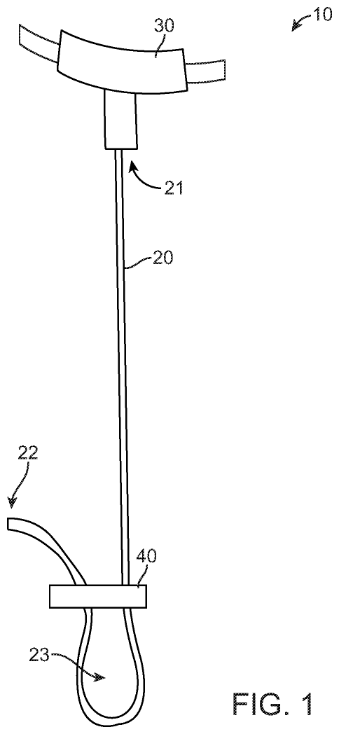

In a first preferred embodiment as seen in FIG. 1, the present invention comprises a wellness device system 10 for maintaining a sleeper in a non-supine position, comprising: a length of elastic material 20; a cuff 30 attached to a first end 21 of the length of elastic material 20; and a clamp 40 securing a second end 22 of the length of elastic material 20 to a mid-section of the length of elastic material, thereby forming a loop 23 of elastic material at the opposite end to the wrist cuff 30. In this embodiment, clamp 40 may be removable and repositionable to adjust the length of loop 23.

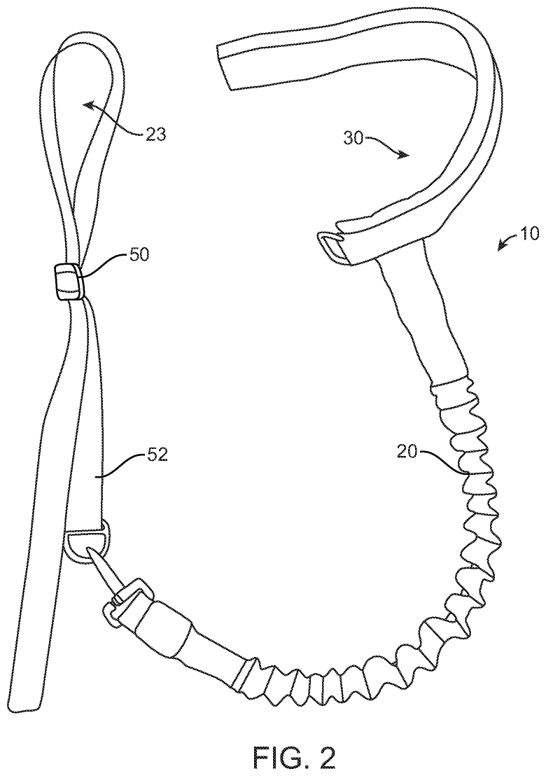

Alternatively, as seen in FIG. 2, the clamp may be replaced with a strap of material 52 connected to the end of elastic material 20. Strap of material 52 preferably passes through a buckle 50 (thereby adjusting the size of loop 23). In this embodiment, the end of strap 52 is simply looped back over itself and fed through buckle 50 to produce loop 23. It is to be understood, however, that the present invention is not so limited and that other clamping or bucking or fastening systems can be used. The length of elastic material may be formed from an elastic tube, a band of elastic material, a tension exercise band, etc. Optionally, the elastic material may be made of latex or synthetic rubber or other suitable materials. In further preferred aspects, the user selects the tension strength of band 20 by selecting one of a set of exercise bands or tubes having different tension strengths.

In operation, cuff 30 is positioned around the wrist of the user's upwardly-facing arm (i.e. with the user resting in a non-supine side-laying position. Preferably, cuff 30 is a padded wrist cuff that has a diameter that is adjustable by the user (to accommodate different users having different wrist sizes). Preferably, cuff 30 is open and closed and adjusted in size by using hook and loop (e.g.: Velcro®) fasteners.

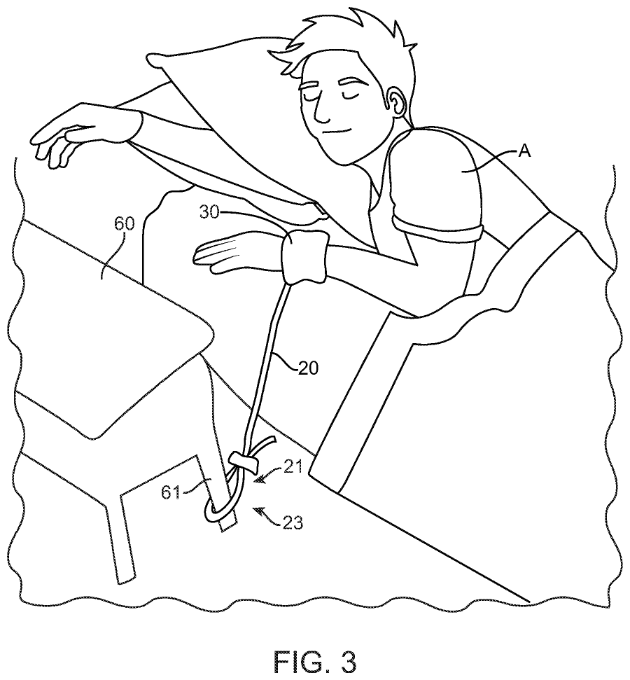

As seen in FIGS. 3 to 7, the present system also includes methods of use and treatment. Referring first to FIG. 3, a method of maintaining a sleeper in a non-supine side-lying sleeping position is provided, comprising: (a) placing a wrist or arm of a sleeper into cuff 30, wherein cuff 30 is connected to first end 21 of a band or tube of elastic material 20; and (b) connecting second end 22 of the band of elastic material 20 to a stationary object (specifically in FIG. 3, the leg 61 of a night table 60). Connecting end 22 of the length of elastic material 20 to leg 61 of night table 60 provides tension on cuff 30, thereby restraining the range of motion of the user's upwardly facing arm A when sleeping; thereby (c) having the sleeper sleep in a non-supine side-lying sleeping position with cuff 30 connected to upper facing arm A, such that the tension on cuff 30 pulls on the user's arm A and thereby prevents the sleeper from rolling onto their back.

Preferred method steps also include adjusting the length of the loop 23 of elastic material 20 to thereby adjust the tension on cuff 30 (for example by removing and repositioning a clamp 40 on the band of elastic material 20, or by threading a different length of elastic material through buckle 50). In optional embodiments, the user selects the band of elastic material from a set of different exercise bands or tubes having different tension strengths.

In operation, achieving a desired amount of tension is beneficial since this helps the user fall asleep without feeling constrained, yet also prevents sufficient tension to prevent the user from rotating onto their back when cuff 30 is on their wrist or arm.

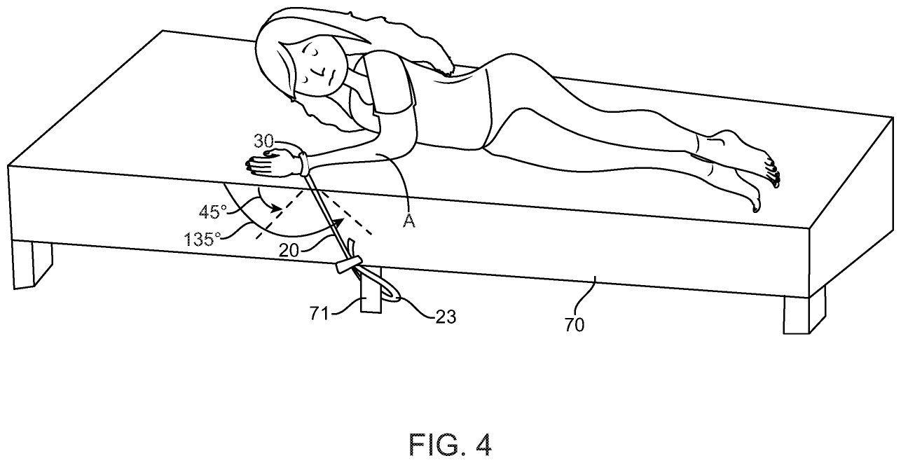

As seen in FIG. 4, the end 22 of elastic material 20 may optionally be secured to a side middle leg 71 of a bed 70 (as opposed to the leg 61 of the night table shown in FIG. 3). In preferred aspects, the band of elastic material 20 is positioned to pull at an angle of 45 to 135 degrees to the side of bed 70. Optionally, however, the band of elastic material 20 may pull at an angle perpendicular to the side of bed 70.

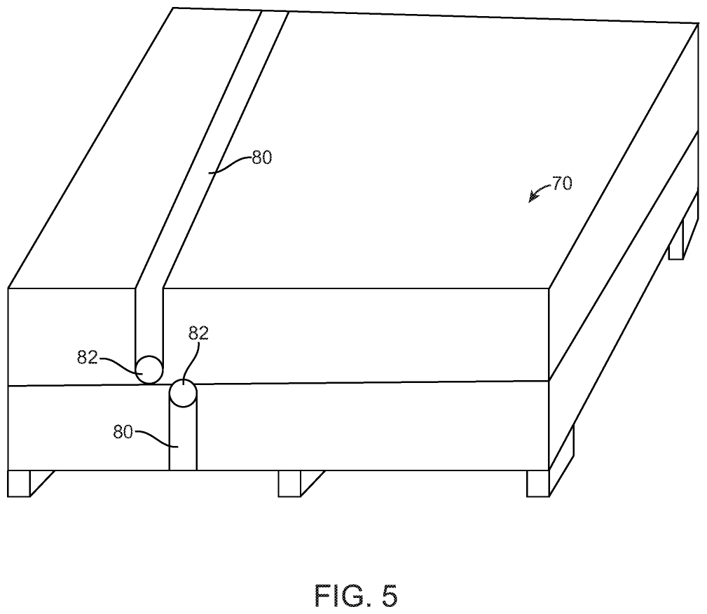

FIG. 5 illustrates a first anchoring mechanism that can be used to secure end 22 of elastic material 20 to a fixed object. An advantage of this approach is that the mechanism has the benefit of providing an anchoring system for end 22 of elastic material 20 when no stationary object is available nearby. An adjustable latex or synthetic rubber band 80 slides around the width of the box spring, mattress, or platform bed (both versions are illustrated for completeness). Band 80 is preferably flat and thin to not create a lump in the sleeping area or otherwise damage bed 70. Band 80 preferably has an eyelet 82 attached thereto through which loop 23 of elastic material 20 can be attached.

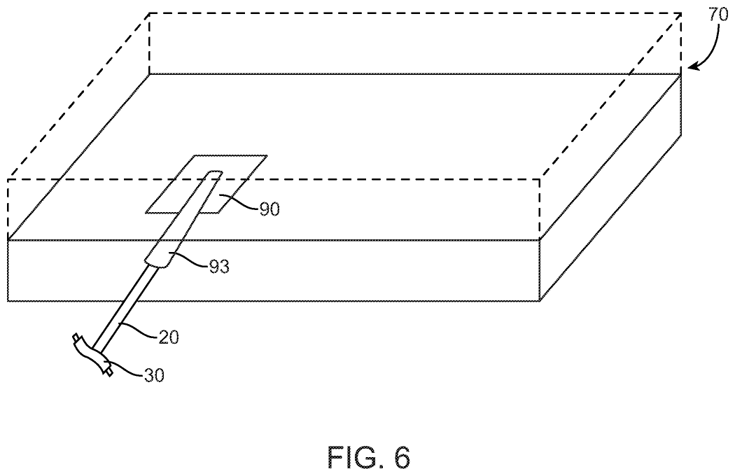

Alternatively, as seen in FIG. 6, a hook and loop style fastener patch 90 can be adhered to the top of the lower box spring portion of bed 70. Another hook and loop style fastener patch can be attached to the bottom of the mattress (shown in dotted lines). The end 22 of the elastic material 20 is then sandwiched between these two patches, thereby holding end 22 in place between the box spring and the mattress of the bed. As such, cuff 30 is held in position.

Optionally, end 22 may be connected to an adjustable strap 93 that is provided to adjust the tension pulling on cuff 30 when it is put onto the wrist or arm of the user's upwardly facing arm. Moreover, the weight of the mattress and user would provide additional assurance that the two hook and loop fastener surfaces would not separate from one another, yet the system can be removed with minimal damage.

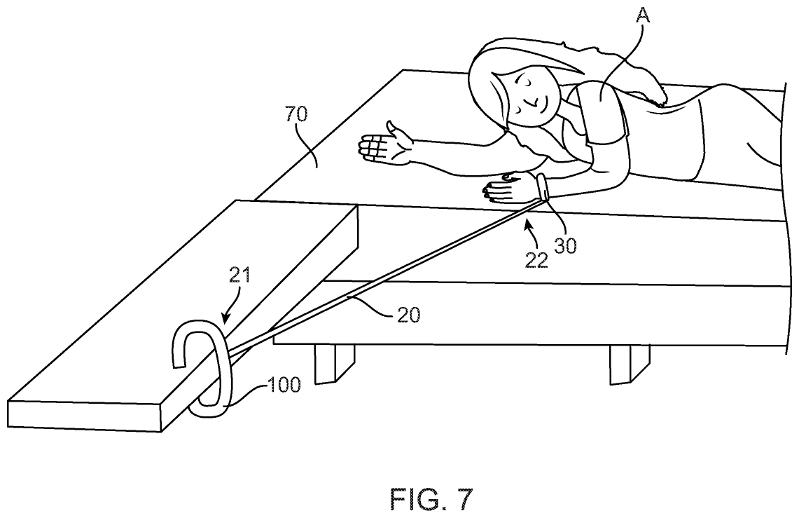

Finally, in the alternative mechanism of FIG. 7, a clamp or hook 100 can be attached to the top of a nearby shelf, book case or other surface. In operation, the user can use this hook or spindle 101 to hold the loop 23 of elastic material 20. The loop 23 of elastic material can be wrapped around the clamp or hook 100 to provide tension to the user's upper arm A as shown. To make the device easy to reach, a clamp with a rubber hook or spindle should be affixed next to the bed with the handle of the device looped over it.

What is claimed:

1. A system for maintaining a sleeper in a non-supine position, comprising:
a length of elastic material;
a cuff attached to a first end of the length of elastic material; and
a loop of material forming an anchor at an opposite end of the length of elastic material to the wrist cuff.

2. The system of claim 1, wherein the loop of material is formed by a clamp securing a second end of the length of elastic material to a mid-section of the length of elastic material, thereby forming a loop of elastic material at an opposite end to the wrist cuff.

3. The system of claim 1, wherein the loop of material is formed by a strap of material attached to the second end of the length of elastic material, and wherein the strap of material passes through a buckle.

4. The system of claim 1, wherein the length of elastic material is an elastic tube or a band of elastic material.

5. The system of claim 1, wherein the non-supine position is a side-lying position.

6. The system of claim 1, wherein the cuff is a padded wrist cuff.

7. The system of claim 6, wherein the wrist cuff is closed by hook and loop fasteners.

8. The system of claim 1, wherein the elastic material is an exercise band made of latex or synthetic rubber.

9. The system of claim 1, wherein the elastic material is selected by the user from a set of different exercise bands having different tension strengths.

10. The system of claim 1, further comprising a hook or clamp for securing the loop of elastic material to a stationary object.

11. The system of claim 1, further comprising:
a first hook and loop fastener patch connected to the loop of elastic material, and
a second hook and loop fastener connected to a bed, such that the loop of elastic material can be secured to the bed.

12. The system of claim 1, further comprising:
a band wrapped around the bed having a connection eyelet attached thereto, wherein the loop of elastic material is secured to the connection eyelet.

13. A method of maintaining a sleeper in a non-supine side-lying sleeping position, comprising:
placing a wrist or arm of a sleeper into a cuff, wherein the cuff is connected to a first end of a band of elastic material;
connecting a second end of the band of elastic material to a stationary object to thereby provide tension on the cuff; and
having the sleeper sleep in a non-supine side-lying sleeping position with the cuff connected to the upwardly facing arm, wherein the tension on the wrist cuff prevents the sleeper from rolling onto their back.

14. The method of claim 13, wherein the second end of the band of elastic material is a loop of elastic material.

15. The method of claim 14, further comprising:
adjusting the length of the loop of elastic material to thereby adjust the tension on the wrist cuff.

16. The method of claim 13, further comprising:
having the user select the band of elastic material from a set of different bands having different tension strengths.

17. The method of claim 13, wherein connecting a second end of the band of elastic material to a stationary object comprises:
connecting the loop of elastic material around a hook or clamp connected to a stationary object.

18. The method of claim 13, wherein the band of elastic material pulls at an angle of 45 to 135 degrees to the side of the bed.

19. The method of claim 18, wherein the band of elastic material pulls at an angle perpendicular to the side of the bed.
